(19) United States
(12) Plant Patent Application Publication
(10) Pub. No.: US 2026/0372222 P1
(43) Pub. Date: Nov. 19, 2026

(54) Delphinium Variety Named 'ET DLP 818'

(50) ;

(71) Applicant: Eternal Plant Boijl B.V., Boijl (NL)

(72) Inventors: Elisabeth Bisschop, Boijl (NL); Bart Noordhuis, Boijl (NL)

(21) Appl. No.: 19/677,491

(22) Filed: May 14, 2026

(30) Foreign Application Priority Data
May 16, 2025 (QZ) 20251036

(51) Int. Cl. A01H 6/72; A01H 5/02

(52) U.S. Cl. CPC A01H 6/72; A01H 5/02
USPC PLT/423

(57) ABSTRACT

A new and distinct cultivar of Delphinium plant named ‘ET DLP 818’, having continuous and abundant flowering with blue-purple color.

Botanical Classification:

Delphinium Elatum L.

Varietal denomination:

‘ET DLP 818’.

### CROSS REFERENCE TO RELATED APPLICATIONS

The present application claims priority to European Community Plant Variety Rights Application No. 2025/1036, filed May 16, 2025, which is herein incorporated by reference.

### BACKGROUND OF THE INVENTION

The present invention relates to a new and distinct cultivar of Delphinium, botanically known as Delphinium Elatum L., and hereinafter referred to by the name ‘ET DLP 818’

The new Delphinium was discovered by the Inventor on Jul. 14, 2018, in a controlled environment in Boijl, Netherlands. The new Delphinium was selected on the basis of abundant flowering.

Asexual propagation of the new cultivar by tissue culture since Mar. 3, 2019, in Boijl, Netherlands, has shown that the unique features of this new Delphinium are stable and reproduced true to type in successive generations.

### SUMMARY OF THE INVENTION

The following traits have been repeatedly observed and are determined to be unique characteristics of ‘ET DLP 818’. These characteristics in combination distinguish ‘ET DLP 818’ as a new and distinct cultivar:
1. Continuous and abundant flowering.
2. Very strong flower stems.
3. Self-branching flower stems.
4. Many compact leaves, and relatively short internodes.
5. Very strong plant habit.
6. Blue purple color.

In side-by-side comparisons conducted by the Inventor, plants of the new Delphinium differed from plants of the parent, the Delphinium cultivar ‘ET 98’, in the following characteristics:
7. Claimed plant is shorter than the parent plant.

In side-by-side comparisons conducted by the Inventor, plants of the new Delphinium differed from plants of the parent, an unidentified variety of Delphinium crossed with Belladonna, in the following characteristics:
1. Claimed plant is shorter than the parent plant.

Plants of the new Delphinium can be compared to the plants of the Delphinium cultivar ‘ET DLP 17 03’, patented as U.S. Plant Pat. No. 33,009. In side-by-side comparisons conducted by the Inventor, plants of the new Delphinium differed from plants of the cultivar ‘ET DLP 17 03’ in the following characteristics:
1. Claimed plant has a shorter flowering time.
2. Claimed plant has a more blue-purple color.

### DESCRIPTION OF THE DRAWINGS

The accompanying colored photographs illustrate the overall appearance of the new Delphinium, showing the colors as true as it is reasonably possible to obtain in colored reproductions of this type. Colors in the photographs may differ slightly from the color values cited in the detailed botanical description, which accurately describes the colors of the new Delphinium.

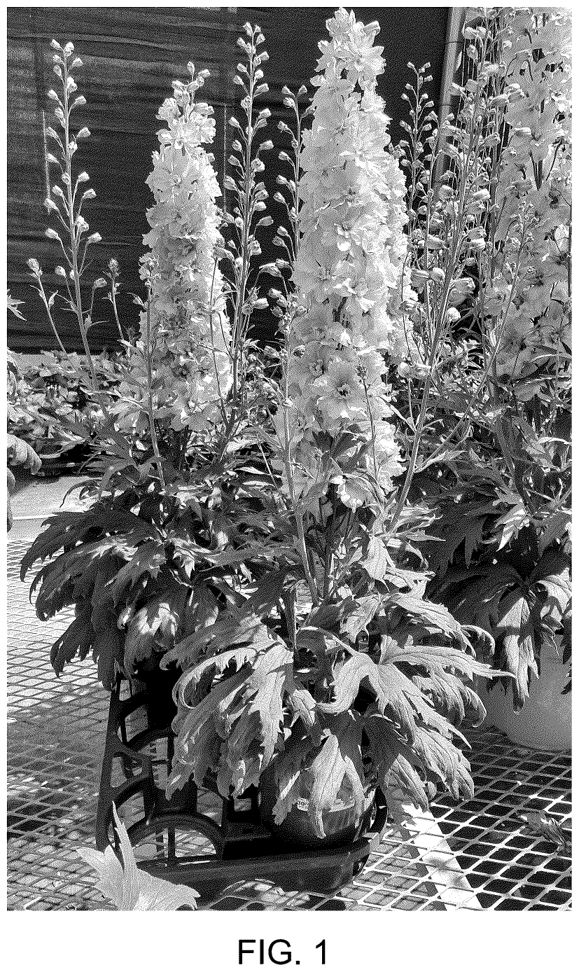

The photograph of FIG. 1 comprises a side view of a typical plant of ‘ET DLP 818’.

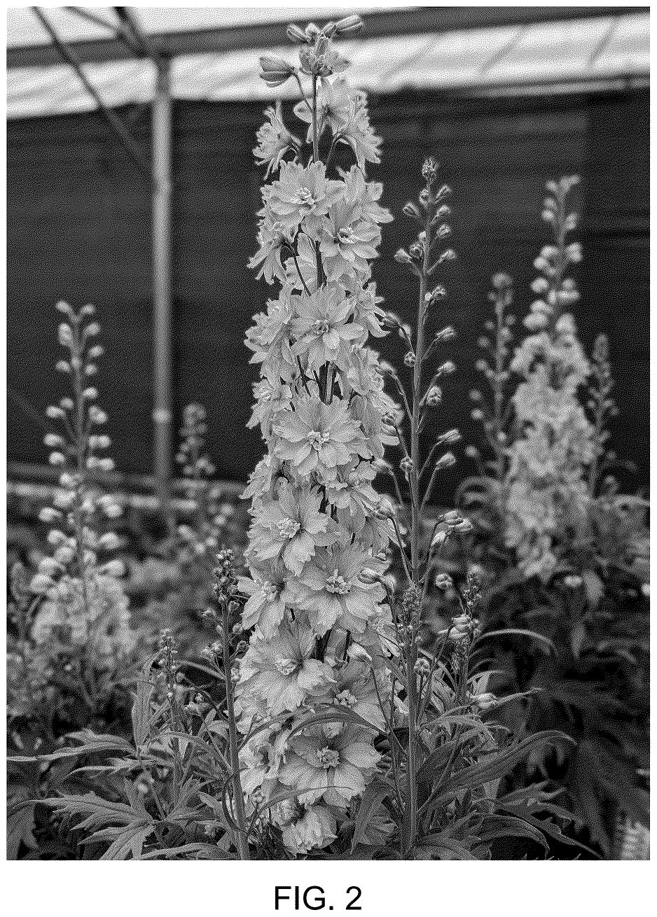

The photograph of FIG. 2 comprises a close-up view of a typical plant of the new Delphinium.

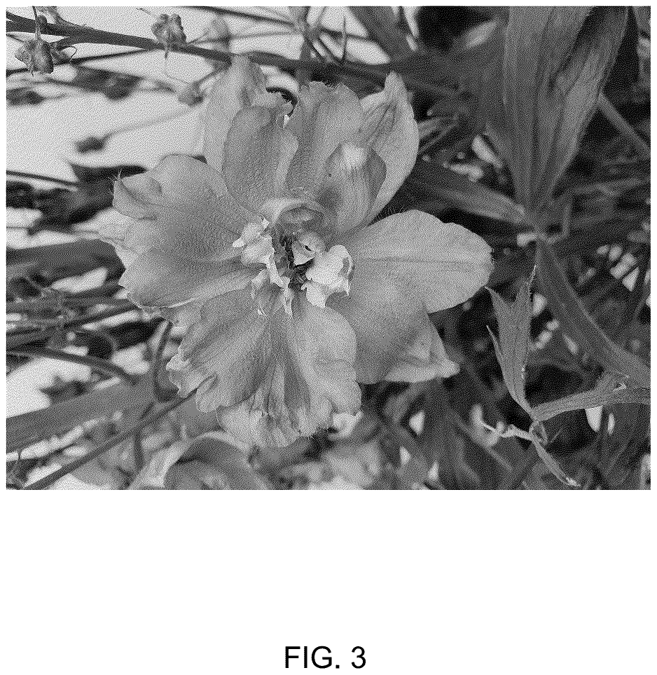

The photograph of FIG. 3 comprises a close-up view of typical flowers of the new Delphinium.

### DETAILED DESCRIPTION

The cultivar Delphinium has not been observed under all possible environmental conditions. The phenotype may vary somewhat with variations in environment such as temperature and light intensity without, however, any variance in genotype.

The aforementioned photographs and following observations and measurements describe plants of the new Delphinium that were grown in a nursery in Boijl, Netherlands. Plants used for the photographs and description were about one year from planting. Color references are made to The Royal Horticultural Society Colour Chart, 2001, except where general terms of ordinary dictionary significance are used.

### PLANT

Botanical classification: Delphinium Elatum L.
Parentage: Delphinium cultivar ‘ET 98’ and an unidentified variety of Delphinium crossed with Belladonna
Propagation:
   Time to initiate roots.—Summer: 12-16 days at 15-21 degrees Celsius. Winter: 15-25 days at 5-10 degrees Celsius.
   Time to develop roots.—Summer: 4 weeks at an average of 20 degrees Celsius. Winter: 5-6 weeks at an average of 12 degrees Celsius.
   Time to produce a finished flowering plant.—Summer: 9 weeks at 18-20 degrees Celsius.
   Root description.—Moderate density and freely branching.
Plant description:
   Form.—Upright and compact.
   Growth rate/vigor.—Medium to strong.
   Plant height.—Up to 80 cm.
   Plant width.—35-45 cm.
   Foliage.—Arrangement: Alternate. Number of Leaves per Stem: 4-6. Length: 12-14 cm. Width: 16-22 cm. Young leaves: Upper surface color: 137B. Lower surface color: 138B. Fully expanded leaves: Upper surface color: 139A. Lower surface color: 138A.
   Venation.—Type: Pinnate to Palmate Color: Upper surface: 136A Lower surface: 137B.
   Petiole.—Length: 4-6 cm (young) and 6-8 cm (mature) Diameter: 2-4 mm (young) and 4-6 mm (mature) Texture: Smooth and pubescent Color: Light green RHS 144 A.
   Lateral Leaflets.—Length: 12-14 cm Width: 3-5 cm Shape: Palmately lobed Shape of Apex: Acute Shape of Base: Obtuse to cordate Orientation and aspect: Outwards and slightly drooping Texture: Upper surface: Smooth Lower surface: Rough and slightly pubescent Margin: Lobed.
Flowers:
   Bud.—Shape: Oblong Diameter: 1-2 cm Length: 1-3 cm Color: 142C.
   Natural Flowering Season.—Spring-Summer.
   Inflorescence.—
   Type.—Double.
   Longevity.—Between 14 and 20 days.
   Average number per plant.—More than 200.
   Fragrance.—None
   Diameter.—Up to 7 cm.
   Height.—2-4 cm.
   Petals.—Number per inflorescence: At least 10-14Shape: More obovate than elliptic Length: 2-4 cm Width: 2.5 cm Shape of Base: Attenuate Margin: Entire/Undulate and folded Texture: Upper surface: Smooth, satiny Lower surface: Smooth, satiny Color: When opening: Upper surface: 91A in the middle of one or more single flower; Some small petals grow 155A with vertical 91A stripes Lower surface: 91A with vertical 142B stripes At maturity: Upper surface: 91A in the middle of one or more single flower; Some small petals grow 155A with vertical 91A stripes Lower surface: 91A with vertical 142B stripes.
Reproductive organs:
   Anther.—Apex Shape: Rounded Base Shape: Oblong Length: 1-2 mm Width: 1-2 mm Color: Edge: 148A but more gray Center: 148A but more gray.
   Stigma.—Shape: Rounded Color: 155D
   Style.—Length: Unable to Observe Color: Unable to Observe
   Ovary.—Length: 2 mm Color: 143B.
Disease/pest resistance:
   Disease resistance.—Normal.
   Pest resistance.—Normal.
   Weather tolerance.—Temperature tolerance: Normal Drought tolerance: Normal.

It is claimed:

1. A new and distinct cultivar of Delphinium plant named ‘ET DLP 818’, as herein illustrated and described.
